(19) United States
(12) Patent Application Publication
(10) Pub. No.: US 2014/0329204 A1
(43) Pub. Date: Nov. 6, 2014

(54) DURABLE COATING METHOD FOR ENHANCEMENT OF SURFACE SCANNING OF DENTITION

(71) Applicants: Duane Milford Durbin, San Diego, CA (US); Dennis Arthur Durbin, Solana Beach, CA (US)

(72) Inventors: Duane Milford Durbin, San Diego, CA (US); Dennis Arthur Durbin, Solana Beach, CA (US)

(21) Appl. No.: 13/888,253

(22) Filed: May 6, 2013

(51) Int. Cl. A61K 6/10; A61C 9/00

(52) U.S. Cl. CPC A61K 6/10; A61C 9/0053
USPC 433/214; 433/217.1

(57) ABSTRACT

A method is provided for enhancing the results of an optical scan of the surface of an object such as human dentition in which the surface may be dry or wet. The method includes the step of applying a composition to the surface, wherein the composition includes a volatile component that rapidly evaporates resulting in the transformation of the residual composition to a durable, water insoluble, non sticky film on the surface, whether applied in a dry-field or in a wet-field environment. The composition generally includes an agent that improves the quality of the optical return signal to a 3D optical scanner. The method further includes the steps of scanning the surface having the film with an optical scanner and then, once scanning has been completed, using an organic solvent, such as a mouthwash containing alcohol, to remove the film from the object's surface.

### BACKGROUND

Digitally based 3 dimensional (3D) computer aided design and computer aided manufacturing (CAD-CAM) is becoming an increasingly effective method of designing and fabricating dental prosthesis. The digital data representing the 3D contour of the dental patient's dentition that is input to the CAD-CAM system at the start of the process can be accomplished in several ways: 1) a traditional flexible impression can be taken and a stone model cast from the impression can be digitized utilizing several types of scanning approaches; 2) the original impression alone can be electronically digitized; 3) the patient's teeth surfaces can be directly digitized in-situ, intra-orally. The intra-oral digital impression approach has a number of advantages such as improved accuracy over traditional cast stone models due to elimination of shrinkage from the impression material. Another advantage is a reduction in time for the whole impression to digital model file process. Elimination of the waste of the casting and impression materials is yet another advantage of a direct intra-oral digital impression.

Several forms of inter-oral 3D optical digital impression systems currently exist and their method of operation is known in the art and described in patent literature. For reference see OraMetrix, Inc., publication no. WO 01/80761, Rekow, et al., U.S. Pat. No. 5,027,281, Massen et al., U.S. Pat. No. 5,372,502, Durbin, et al U.S. Pat. No. 6,386,876 and Brandestini et al., U.S. Pat. No. 4,837,732. These systems utilize some form of optical imaging of the tooth surface which generally requires the translucent optical properties of the tooth surface to be covered with a light scattering and opaquing coating containing an agent such as titanium dioxide (TiO₂). This coating ensures that the optical return signal being measured from the tooth surface is from the outer surface of the tooth and not a false signal from an internal reflection or scattered signal from the internal tooth structure. In addition, the light scattering properties of the coating generally increase the amount of light in the optical return signal which can result in a higher quality 3D image of the scanned surface. Although several optical based surface data capture approaches are being utilized in the currently commercialized scanning devices, scanning the dentition using a structure light approach is among the fastest and most accurate. With this approach, the scanning device projects a pattern towards the surface of interest, optically observes the distortion in the pattern in the optical signal returned from the surface and computes the surface topography, i.e. the 3D surface contour, from the distortion of the pattern in the returned optical signal. For certain non-homogeneous surfaces of interest there can be a substantial variation in the inherent optical signal return from different portions of the surface such that the projected pattern from a scanner will reflect or scatter back some portions of the projected pattern onto the imaging device of the scanner to a satisfactory degree without any prior preparation of the surface. Other portions of the surface may be relatively non-reflective, non-scattering or translucent, and therefore do not provide an adequate optical signal return of the projected pattern back to the scanner's imaging sensor. For such surfaces, the scanning of the object can be improved by applying a substance to the surface to improve the quality of the optical return signal observed by the scanner's imaging sensor.

To address this problem of inconsistent optical properties of the surface of interest, the CEREC scanning device developed by Brandestini et al. is used in conjunction with a TiO₂-based aerosol powder which is applied to the teeth. When the object being scanned is the TiO₂ coated teeth, gums, or other intra-oral structures of a human patient, a new problem arises in that the TiO₂-based aerosol powder used in the CEREC system is readily washed away by saliva and also tends to form clumps and a slurry like texture when exposed to moisture in the intra-oral cavity. In addition, this coating does not adhere well to the dentition and is easily wiped off by any inadvertent touching of the coated surface. Voids in the coating of the surface whether due to being rinsed off by saliva, or wiped off by touching, result in corresponding voids in the 3D image captured by the scanner. The overall success of this coating method is largely dependent on the maintenance of a dry-field (i.e. minimal moisture in the intra-oral cavity), the skill of the person applying the coating and, even for skilled personnel, the method becomes very difficult to use successfully for cases where the complete jaw is to be coated and scanned.

To address this shortcoming in the CEREC method of using a TiO₂-based aerosol powder to coat the teeth, OraMetrix U.S. Pat. No. 6,854,973, by Butcher, et al describes a method of coating wherein a binder that reacts with moisture is added to the coating in such a manner that after the binder's reaction with moisture, the binder helps a reflective pigment contained in the coating adhere to a wet-field surface. As taught in the OraMetrix patent, the coating composition comprises a base, a pigment and a binder composed of a material having a tendency to harden or become sticky in the presence of water or saliva and thereby not be washed off the surface of the anatomical structure. The described method continues with the steps of allowing the composition to dry to a film covering the surface of the anatomical structure with the film improving the opacity of the surface, and scanning the surface having the film with a scanner.

While the method taught in the OraMetrix patent does, to some degree, address the challenge of applying a coating to a wet-field surface, it is not an optimal solution to the general problem of applying a coating to the intra-oral dentition of a dental patient in preparation for a 3D optical scan. As taught in the OraMetrix method, the binder in the coating requires a wet-field, i.e. the presence of moisture or saliva on the surface being coated, in order for the binder to harden or become sticky. The OraMetrix method specifically teaches the use of denture adhesives containing Polyvinyl Methyl Ether Maleate (PVMEM) as a suitable binder for use in a wet-field environment. It is known in the art that PVMEM is a chemical that polymerizes in the presence of water (or saliva) thus forming the sticky reaction product described in the OraMetrix method. Further, it is known in the art that the resultant sticky polymerized PVMEM reaction product is also soluble in water. Consequently, while moisture or saliva is initially required to transform the PVMEM into the sticky film, continued exposure to that same moisture and saliva will dissolve, erode and weaken the film to the point where the coating can be prematurely removed from the dentition surfaces before the scanning has been completed. The missing coating results in voids and inaccuracies in the 3D scan data.

A more optimal method for coating dentition in preparation for a 3D optical scan would be a method that is equally effective to apply and use in both a dry-field and a wet-field environment, i.e. the method does not require a wet-field to become activated or a dry field for the coating to stay in place after it is applied. Further, an optimal method would provide a coating that after application is not sticky, and is robust against removal by touching or by even excessive moisture such as dripping or pooled saliva, thus allowing the 3D scanner operator to capture a complete and accurate scan without missing surface data and without the need to continually stop scanning and touch up the coating on surfaces where the coating has prematurely been wiped or washed away in the middle of the scanning process. Further, a number of commercial 3D scanners are intended to be used by resting the scanner's optical window directly on the dentition. In the case of the OraMetrix method, where the coating is sticky, the sticky coating easily transfers from the dentition onto the scanner window where it then blocks the path of the optical return signal. This necessitates the operator's vigilance to watch for the transfer of coating onto the scanner window and then stopping the scanning to clean the window and inspect and touch-up the coating in the area of surface where the sticky coating transfer took place.

### SUMMARY

A method is provided for coating the dentition of a dental patent in preparation for taking an impression of the dentition with a 3D optical scanner. The method is equally suitable for coating dry dentition or dentition that is wet with moisture and salvia. The method is applicable to coating a small portion of the patient's dentition such as a single tooth, or a large portion of the dentition such as the entire arch and the associated tissue. The method includes the step of applying a composition to the dentition, wherein the composition is characterized in that it rapidly transforms to a durable, non sticky film on the dentition whether applied in a dry-field or in a wet-field environment. Once transformed into the durable, non sticky film, the composition is resistant to removal by touching, is water insoluble and maintains a strong adhesion to the dentition in the presence of a prolonged exposure to moisture and saliva. The composition generally includes an agent that renders the coated surface to be substantially opaque or otherwise improves the optical properties of the coated surface such to increase the quality of the optical return signal to a 3D optical scanner. The method includes the step of scanning the surface having the composition applied thereto. The method further includes the step of using a biocompatible agent to remove the coating film from the dentition after the 3D scan has been completed.

A number of suitable formulations for a durable coating composition are possible. One is a liquid composition which includes a volatile liquid base, such as dehydrated ethanol, a reflective pigment, and a film forming agent dissolved in the alcohol that works in both a dry-field and a wet field environment and which promotes a durable, non sticky adhesion of the coating to the surface of the patient's dentition.

A suitable film forming agent for incorporating into a coating composition for scanning dentition is the family of thermoplastic cellulose ethers. Thermoplastic cellulose ethers are derived from cellulose and have the same chemical structure as this naturally occurring polymer. This class of water insoluble, organosoluble polymers function as tough, flexible, non-sticky film formers that once formed are extremely durable and stable, even in the continuous presence of liquid water and saliva. Thermoplastic cellulose ethers are colorless, odorless, tasteless and inert to attack by water or water based agents such as saliva. Thermoplastic cellulose ethers are biocompatible, classified as generally recognized as safe, and routinely used in a variety of pharmaceutical, food and personal care products.

Thermoplastic cellulose ethers are readily soluble in organic volatile solvents, including ethanol, and can be formulated to provide a range of viscosities in ethanol while retaining excellent film forming characteristics. For example, a coating composition that is intended to be applied by brush may use a higher viscosity formulation of thermoplastic cellulose ethers dissolved in a volatile solvent, while a coating composition intended to be applied by spraying may used a lower viscosity formulation of thermoplastic cellulose ethers dissolved in a volatile solvent.

A solution of thermoplastic cellulose ethers dissolved in a liquid volatile solvent, mixed with a pigment and applied to a surface does not depend upon the surface being a dry-field or a wet-field in order for the adhesion and film forming characteristics of the thermoplastic cellulose ethers to take place. After being applied to a surface, the liquid volatile solvent begins to evaporate and as it does so, the concentration of the dissolved thermoplastic cellulose ethers dissolved in the remaining liquid solvent increases to a critical threshold concentration level as the liquid volatile solvent evaporates and, once this critical concentration level is reached, the thermoplastic cellulose ethers rapidly drops out of solution and forms a durable, non-sticky, water insoluble, polymer film on the coated dentition. The pigment contained in the composition is trapped in the film which promotes the binding of the pigment on the surface and provides the desired optical reflection and opacity characteristics for the coated surface. The formed film of the coating composition remains soluble in organic solvents and therefore, after the scanning of the dentition has been completed, the coating may be removed with a simple organic solvent rinse, for example by rinsing using a mouthwash containing alcohol.

While the above disclosure is based on a formulation using thermoplastic cellulose ethers as the film forming agent, other suitable compositions can be derived by persons skilled in the art from the teachings disclosed herein.

The present invention contemplates the use of a liquid composition applied either using a brush or as an aerosol depending on the preference of the user. In either case the composition may be applied with equal effectiveness to both dry-field and wet-field surfaces whereby, after the application, the film forming agent rapidly binds the pigment in the composition to the coated surface in a durable, non-sticky water insoluble film and the surface is ready to be scanned.

In one embodiment of the invention, a method of scanning a surface, such as the in-vivo scanning of the tissue of a human, is provided in a dry-field or a wet-field environment, comprising the step of first applying a biocompatible composition to the surface before scanning For example, the composition may comprise a volatile liquid base, a pigment and a film forming agent composed of a material that is initially dissolved in the volatile base but is formulated in a manner such that after the composition is applied to the surface, the liquid volatile base will evaporate and the concentration of the binder material dissolved in the remaining volatile liquid base will increase to a threshold concentration at which point the binder has the tendency to drop out of solution and form a durable film on the coated surface. The absence, or the presence, of water or saliva on the surface that the composition is applied to does not affect the film forming characteristics of the composition. The method continues with the steps of allowing the volatile liquid base in the applied composition to evaporate to a level where the concentration of the film forming binder material in the remaining liquid base exceeds a threshold that results in the rapid formation of a film that binds the opacity improving pigment in the composition to the coated surface, scanning the surface having the film with a 3D optical scanner and, after the scanning is complete, removing the water insoluble film and pigment from the coated surface with a biocompatible organic solvent that dissolves the film.

The present invention provides methods which are well suited for scanning surfaces such as intra-oral dentition, including scanning a single tooth, a set of teeth, the entire upper and/or lower jaw, the gingiva, or the palate of a human whether the surface presents in a wet-field or a dry-field environment. The non-sticky, tightly adhered, water insoluble and durable film formed by the present invention on the coated dentition facilitates scanning with a variety of commercial 3D optical scanners, including scanners that employ an optical window that can rest directly on the coated dentition surface. Further, the methods can be used to perform 3D scanning of orthodontic and dental appliances and devices, such as orthodontic brackets, crowns, partials, dentures and other devices. Finally, while the methods are particularly useful for 3D optical scanning of in-vivo tissue such as dentition, the methods are also applicable to other types of optical scanning situations in which the user desires to improve the optical properties of the surface to be scanned by applying a durable, non-sticky, water insoluble, but easily removable coating to the surface of interest before scanning

### DETAILED DESCRIPTION

The quality of the result from 3D optical scanning of certain surfaces such as the human dentition generally is improved by first applying a coating to the dentition to improve the optical properties of the coated structures. Without first being treated with a coating, intra-oral scanning of dentition surfaces is difficult due to the variety of the surfaces and the range of optical properties of these surfaces. For example the intra-oral dentition surfaces of interest for 3D optical scanning may include natural teeth with enamel, natural teeth with the enamel removed, gums, the palate, teeth previously treated and filled with amalgonate or gold fillings, PFM crowns, ceramic crowns, implants and artificial teeth. In an orthodontic treatment setting, the dentition may be covered with brackets and wires that create further challenges for successful 3D optical scanning The success of performing a 3D optical scan on such a range of disparate surfaces, with their wide differences in optical properties is improved when the surfaces are first treated with a coating that provides a more homogeneous surface for reflecting an optical return signal back to the 3D scanner's image sensor.

The prior art is based on either: 1) applying a opaquing formulation in a dry-field environment, such as the TiO₂ powder used in the CEREC method discussed previously, and wherein the dry-field must substantially be maintained throughout the scanning process; or 2) applying an opaquing formulation in a wet-field environment, such as the OraMetrix method discussed previously, wherein the moisture in the wet-field is required to activate the binder in the formulation and form the sticky film used to bind the opaquing agent. In contrast to the prior art, the present invention does not depend upon the absence (dry-field) or the presence (wet-field) of moisture on the surface being coated in order to form a non-sticky, durable coating that is inert to attack by water or saliva. Further the present invention results in a coating that remains inert to attack by water or salvia during the scanning process, with the coating maintaining a durable film until it is removed after the scanning is complete using a biocompatible organic solvent such as ethanol.

In a first aspect, the present invention provides for scanning the surface of an object in which moisture such as saliva or water may, or may not, be present on the surface. The method comprises the steps: 1) applying a composition to the surface, wherein the composition is characterized in that it rapidly forms a non-sticky, durable film that is inert to attack by water or saliva; 2) scanning the surface having the composition applied with a 3D optical scanner; and 3) removing the coating film from the surface using a biocompatible organic solvent such as ethanol.

In another aspect, the present invention provides an opaquing fluid for application to a surface of an object in preparation for 3D optical scanning of the object, comprising, in combination: a biocompatible, liquid composition comprising a mixture of a volatile liquid base, a pigment, and a binder composed of a material that is dissolved in the volatile base to form a solution. When the concentration of the binder material dissolved in the volatile liquid base exceeds a concentration threshold, the dissolved binder material drops out of solution and forms a durable, non-sticky, water insoluble film. The volatile liquid base comprises a substantially non-aqueous liquid which rapidly vaporizes when applied to a wet or a dry surface and, as the volatile liquid evaporates, the concentration of the binder in the remaining volatile liquid base on the surface exceeds a concentration threshold, at which point the binder drops out of solution and forms a durable, non-sticky, water insoluble film on the surface. The pigment in the opaquing fluid is held on the surface by the film forming binder and results in a film on the surface that is substantially opaque.

In a preferred possible embodiment of the present invention, the composition comprises a liquid composition. The composition can be applied to the surface by means that include brushing, spraying or dipping. The method taught in this present invention is suitable for preparing an anatomical structure of a human wherein the anatomical structure may comprises oral structures such as lips, gingival tissue, teeth, etc. and where the surface of such structures may be wet or dry. In one embodiment, the liquid composition is comprised of a volatile liquid base of ethanol alcohol into which is dissolved thermoplastic cellulose ethers. The composition further is comprised of a pigment such as, but not limited to, Titanium Dioxide (TiO₂) that serves as an optical reflecting or opaquing agent on the surface the composition is applied to. Depending on the characteristics of the specific pigment used, the pigment may be dissolved completely in the base, or, alternatively, the pigment may be suspended or emulsified in the base in which case the pigment may float or otherwise be suspended in the liquid base. The viscosity of the liquid composition can have a range of values, including low viscosity formulations for application by spraying and higher viscosity formulations for application by brushing or dipping. A representative formulation is now described that has been found to be superior in terms of functionality and ease of use as compared to a prior art formulations. The formulation comprises a liquid phase, biocompatible composition that includes 1) a liquid base, such as a liquid alcohol base, e.g. ethanol, 2) a pigment such as TiO₂, and 3) a binding agent comprised of thermoplastic cellulose ethers at a concentration such that the binding agent is initially dissolved in the liquid base. The concentration range for a binder in the formulation will vary depending the binder and on the nature of the object being scanned, and for example can be present in the range of between 5% and 25% by weight. While the pigment in a preferred embodiment incorporates TiO₂ as an opaquing agent, other pigments are possible, such as zinc oxide or other agents having some tint, color or other property that improves the optical return signal from a coated surface back to a 3D optical scanner's image sensor. The pigment is preferably present in the formulation in the range of between 5 and 25 percent by weight. Some formulations may include deionized water added to the liquid composition in order to tailor the viscosity of the final composition.

The formulation set forth below: 1)provides good adherence to the dentition's surface; 2) can be applied with equal effectiveness to surfaces that are wet or dry; 3) promotes the rapid formation of a uniform, thin film less than 100 microns thick on the coated surface and thus does not obliterate the detail in the dentition surface being scanned; 4) is inert to attack by water or saliva during the scanning process; and 5) is easily removed using an alcohol based solution such as mouthwash after the scanning has been completed.

A presently preferred formulation of a coating composition to apply to a surface in preparation for a 3D optical scan is as follows:

| | | Preferred Percent by |
|---|---|---|
| | | Weight for Scanning |
| Description | Percent by Weight Range | Dentition |
| Liquid Base: Dehydrated | 50%-97% | 70% +/− 5% |
| Ethanol 200 Proof (>99% | | |
| ethanol) | | |
| Deionized water | 0%-25% | 10% +/− 2% |
| Binder: thermoplastic | 3%-25% | 5% +/− 1% |
| cellulose ethers powder | | |
| Pigment: TiO₂ powder | 5%-25% | 15% +/− 5% |

The above formulation provides a coating composition that is a white, biocompatible opaque liquid at room temperature. The coating composition is preferably applied by spraying the formulation onto a surface before scanning but it can also be applied by bushing or dipping. After application to a wet or dry surface, the formulation rapidly forms a durable, water insoluble film on the coated surface. The pigment in the coating improves the optical properties of the coated surface and results in the coated surface providing a stronger optical return signal during 3D optical scanning After scanning, the coating film can be quickly removed using a biocompatible alcohol solution such as a mouthwash containing at least 15% alcohol by weight.

Thus, we have invented a method of 3D optically scanning the surface of an object in a dry-field or a wet-field environment comprising the steps of 1) applying a biocompatible composition to the surface; 2) allowing the composition to form a durable, non-sticky film covering the surface, with the film being inert to attack by moisture and the film exhibiting opaque properties to improve the optical return signal from the object to be scanned; 3) scanning the surface having the film with a 3D optical scanner; and 4) after the scanning is completed, removing the film using a biocompatible organic solvent. The object in one possible embodiment may be an anatomical structure of a human, such as the dentition.

It is fully expected that persons skilled in the art will be able to derive other suitable durable film forming compositions for application to surfaces in a dry-field or wet-field environment from the present disclosure. The usage of such additional formulations for 3D optical scanning is contemplated as being within the scope of the appended claims.

1. A method of scanning the surface of an object in which said surface may be dry, or said surface may be wet with moisture such as saliva or water, comprising the steps of: a) applying a liquid composition to said surface, wherein said liquid composition comprises a liquid base, a pigment, and a binder for promoting the binding of said pigment to said surface, wherein said binder comprises a material dissolved in said liquid base and said binder having a tendency to drop out of solution and form a durable, non-sticky, water insoluble film when the concentration of said binder material dissolved in said liquid base exceeds a concentration threshold, and wherein said liquid base comprises a substantially volatile liquid which evaporates when applied to said surface and thereby causes the concentration of said binder in said liquid base to exceed the concentration threshold at which point said binder drops out of solution and forms the durable, water insoluble film on said surface; b) scanning said surface with a 3D optical scanner after said liquid composition has been applied and a durable, water insoluble film has formed; and c) removing said water insoluble film formed on said surface using a liquid that contains an organic solvent.

2. The method of claim 1, wherein the formulation of the liquid composition enables an even application of the liquid composition promoting a uniform, durable, water insoluble film covering said surface, said film being substantially opaque.

3. The method of claim 2 wherein said film covering the surface is less than 100 microns thick and thus does not obliterate fine detail in the surface being scanned

4. The method of claim 1, wherein said object comprises the dentition of a human.

5. The method of claim 1, wherein said liquid base comprises a dehydrated ethanol alcohol having at least 99% purity and deionized water, said pigment is titanium dioxide, and said binder is thermoplastic cellulose ethers.

6. The method of claim 5, wherein said liquid base is present in an amount of 73% to 87%, said pigment is present in an amount of 10% to 20%, said binder is present in an amount of 4% to 6%, by weight.

7. The method of claim 1, wherein said liquid that contains an organic solvent is a mouthwash containing at least 15% alcohol by weight.

8. A method of in-vivo scanning of the dentition of a human in a dry field or a wet-field environment, comprising the steps of:
a) applying a biocompatible, liquid composition to the surface of said dentition, wherein said liquid composition comprises a volatile liquid base, a pigment and a binder composed of a material dissolved in said volatile base and said binder having a tendency to drop out of solution and form a durable, water insoluble film when the concentration of said binder material dissolved in said volatile liquid base exceeds a concentration threshold, and wherein said volatile liquid base comprises a substantially non-aqueous liquid which vaporizes when applied to said surface and thereby causes the concentration of said binder in said volatile liquid base to exceed the concentration threshold at which point said binder drops out of solution and forms the durable, water insoluble film on said surface; b) scanning said surface having said liquid composition applied thereto with a 3D optical scanner; and c) after scanning is completed, removing said film from the surface using a liquid containing an organic solvent.

9. The method of claim 8, wherein said volatile liquid comprises a dehydrated ethanol alcohol having at least 99.0% purity present in an amount of between 50% and 97% by weight and water between 0 to 25% by weight, and wherein said binder material is thermoplastic cellulose ethers dissolved in said alcohol present in an amount of between 3% and 25% by weight and said pigment is titanium dioxide present in an amount between 5% and 25% by weight.

10. The method of claim 8, wherein the formulation of the liquid composition enables an even application of the liquid composition that promotes the formation of a uniform, durable, water insoluble film covering said surface, said film being substantially opaque.

11. The method of claim 10, wherein the said film covering the surface is less than 100 microns thick and thus does not obliterate fine detail in the dentition being scanned.

12. The method of claim 8 wherein said liquid that contains an organic solvent is a mouthwash containing at least 15% alcohol by weight.

13. An opaquing fluid for application to a surface of an object in preparation for 3D optical scanning of the object, comprising, in combination:
a biocompatible, liquid composition comprising a mixture of a volatile liquid base, a pigment, and a binder composed of a material dissolved in said volatile base and said binder having a tendency to drop out of solution and form a durable, non-sticky, water insoluble film when the concentration of said binder material dissolved in said volatile liquid base exceeds a concentration threshold, and wherein said volatile liquid base comprises a substantially non-aqueous liquid which vaporizes when applied to said surface and thereby causes the concentration of said binder in said volatile liquid base remaining on said surface to exceed the concentration threshold at which point said binder drops out of solution and forms the durable, non-sticky, water insoluble film on said surface, with said film being substantially opaque.

14. The fluid of claim 13, wherein said volatile liquid base comprises a mixture of dehydrated ethanol alcohol having at least 99.0% purity and deionized water.

15. The fluid of claim 14, wherein said alcohol is present in an amount of between 50% and 97% by weight and wherein said water is present in an amount of between 0% and 25% by weight.

16. The binder of claim 13, wherein said binder is thermoplastic cellulose ethers and is present in an amount of between 3% and 25% by weight
